(19) United States
(12) Patent Application Publication
(10) Pub. No.: US 2006/0258992 A1
(43) Pub. Date: Nov. 16, 2006

(54) Tattoo-removing substance

(76) Inventor: Remo Stoop, Zurich (CH)

Correspondence Address: RANKIN, HILL, PORTER & CLARK LLP, 4080 ERIE STREET, WILLOUGHBY, OH, 44094-7836, US

(21) Appl. No.: 10/572,731

(22) PCT Filed: Jun. 17, 2004
(86) PCT No.: PCT/CH04/00367

§ 371 Date: Apr. 5, 2006

(30) Foreign Application Priority Data
Sep. 24, 2003 (CH) 1628/2003

(51) Int. Cl. A61M 35/00

(52) U.S. Cl. 604/289; 606/186; 424/680

(57) ABSTRACT

Disclosed is a substance for removing tattoos, comprising an aqueous solution of a alkali chloride that is provided with a pH value of less than 7. Said substance allows old tattoos to removed in a simple, fast, and safe manner while largely preventing the skin from turning red or being irritated. Also disclosed is a device or a machine for carrying out the inventive method.

The present invention relates to a tattoo-removing preparation, to a method using this preparation and to an implement with which to carry out this method.

As regards a known method of this kind, a tincture is introduced under the skin using that instrument ordinarily generating the tattoo. The tincture being used contains sulfur powder, potassium chlorate etc., namely substances that may cause local skin inflammations. Moreover previously known methods sometimes fail to accurately position the needle under the skin surface, at that site holding the tattoo dye. This known treatment must be repeated several times to reach all areas of the particular dye dot with the said preparation. Repeating this procedure further increases the danger of skin inflammation on account of the so-called ingredients of said liquid.

An implement already is known to insert said preparation into the skin. Said implement however incurs a number of drawbacks. Setup time when changing the needle sets is too long, the skin punctures lack adequate impact, the puncture depth is uneven, the machine includes a motor requiring connection to the power outlets, entailing a heavy implement difficult to handle, and also electric shocks may occur because a liquid is present.

The objective of the present invention is elimination of the cited drawbacks and also further drawbacks of the state of the art.

This problem is solved with respect to the initially cited preparation by the features of the invention of claim 1.

The advantages offered by the preparation of the present invention are that old tattoos can be removed in simple, rapid and safe manner and that reddening, i.e. irritation of the skin shall be widely avoided.

Claim 13 defines the method of the present invention making use of said preparation.

The implement of the present invention carrying out said method is defined in claim 13.

Sodium chloride was found to be an essentially appropriate alkali chloride. Typically the pH value of the solution being used is less than 4.0, preferably less than 3.4. Typically the pH of the solution is larger than 1.5, preferably larger than 1.8.

In a particular embodiment mode the preparation contains a weak acid having an acid constant 10⁻² mole/ltr<K₁<10⁻⁵ mol/ltr, preferably being a fruit acid, for instance citric acid.

In another embodiment mode the preparation contains juice from such a citric fruit, preferably pineapple juice. Applicable citrus fruits are those belonging to the citrus species, in particular grapefruit, lemons, bitter oranges, oranges, tangerines and limes.

Typically the alkali chloride concentration in the aqueous solution is in the range of 10-20, preferably 12-16.5% by weight.

In a particular embodiment mode, the preparation also may contain an oil, preferably coconut oil or coconut milk. The aqueous, acid solution and the oil may be mixed in a ratio of 3/1 to 1/1, preferably two parts aqueous acid solution and one part oil. Preferably the aqueous, acid solution is a citrus fruit juice and the oil is coconut milk.

In a further embodiment mode, the preparation of the present invention additionally may contain starch, preferably in the form of potato juice.

The said preparation also may contain one or more additives, preferably preservatives, oxidizers, whitening agents or buffers. Appropriate whitening agents for instance are tyrosinase inhibitors, free radicals scavengers, hydroquinone, vitamin C and its derivatives arbutin and glutathione.

The preparation is advantageously formulated for suitable skin application, for instance being a cream, an oil, a foam, a gel, an emulsion, a solution or a patch.

In application, the said preparation is deposited on a tattooed main part. Preferably skin penetration by said preparation is enhanced by appropriate steps, for instance by massaging it into the skin, whereby the said preparation is moved by diffusion to the dye-bearing main layer. Said preparation furthermore may be moved mechanically to the dye to be removed that is situated underneath the skin surface.

Several recipes for the preparation of the invention are listed below.

### EXAMPLE 1

16.5 g cooking salt are dissolved in 100 ml lemon juice. Also 50 ml coconut milk and 15 ml of potato juice are added to the above solution and are thoroughly mixed. The solution so prepared either can be directly applied to the tattoo to be removed or it may be formulated beforehand into an application form suitable for the skin, preferably as a cream, oil, foam, gel, emulsion or patch.

### EXAMPLE 2

| Substance | %-wt |
|---|---|
| Butyric acid | 70.00 |
| Water | 22.38 |
| Cooking salt | 4.50 |
| Soy bean oil | 0.30 |
| Starch | 0.30 |
| Glycerin | 1.20 |
| Carbopol 1342 | 0.06 |
| Triethanolamin | 0.06 |
| Squalane | 0.90 |
| Sorbitanlaurate | 0.06 |
| Uninontan | 0.09 |
| Phenonip | 0.15 |

### EXAMPLE 3

The tattoo-removing preparation contains a citrus fruit juice, pineapple juice having been found optimally effective. Said preparation furthermore contains also coconut milk. Pineapple juice and coconut milk advantageously are employed in the ratio of two parts pineapple juice to one part coconut milk. This mixing ratio may vary within ±10%. Moreover preferably iodized salt is added to the above mixture. From 100 to 200 g, preferably 110 g of the iodized salt are dissolved per ltr of liquid in the mixture of citrus juice and coconut milk.

The mixture so prepared is deposited on a tattooed skin portion into which it is massaged until the removal preparation has penetrated the skin. The active ingredients diffuse into the skin layer containing the tattooing dyes. To attain the desired results, the preparation of the invention should be deposited at least five times on the tattooed skin position.

To facilitate depositing the preparation of the invention onto the skin, a gel or a salve may be admixed to the removal preparation. The proportion of the gel or salve in the removal cream should not exceed 90%-volume. The number of required treatments may be increased depending on the removal-liquid concentration.

The preparation of the invention deposited on the skin is biologically pure and therefore highly compatible with the skin, entailing no side effects. The tattoos being removed without injury to the skin, scar tissue formation is eliminated, nor is pain incurred. Also, treatment can be carried out at home.

An implement with which to carry out the method of the present invention comprises a tool introducing the preparation of the invention into the dyed skin layer. This tool comprises a set of needles and a support for them. The needles are configured in mutually spaced manner and are connected to one another. The mutually connected needle ends are affixed to the said support and the said tool is connected in such manner to a drive component that this element may be rapidly and simply exchanged with another tool.

The drive component comprises a drive unit and an elongated transmission element which is connected at one end to the drive unit and at the other end to the exchangeable tool.

The transmission element is advantageously a connecting rod; the tool needle support comprises a bolt which may be connected for instance by a bush to one end of the connecting rod.

Illustratively embodiment modes of the present invention are discussed below in relation to the appended drawings.

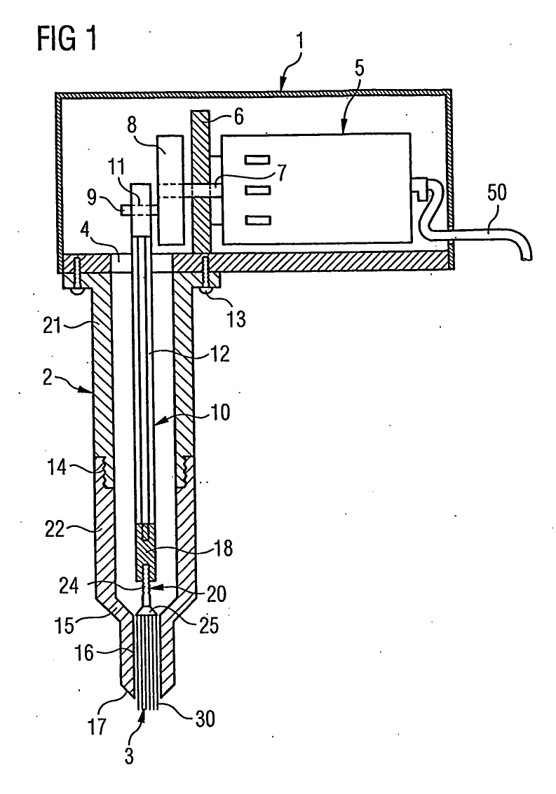

FIG. 1 is a vertical and longitudinal section of a first embodiment mode of one of the means with which the carry out the present method, and

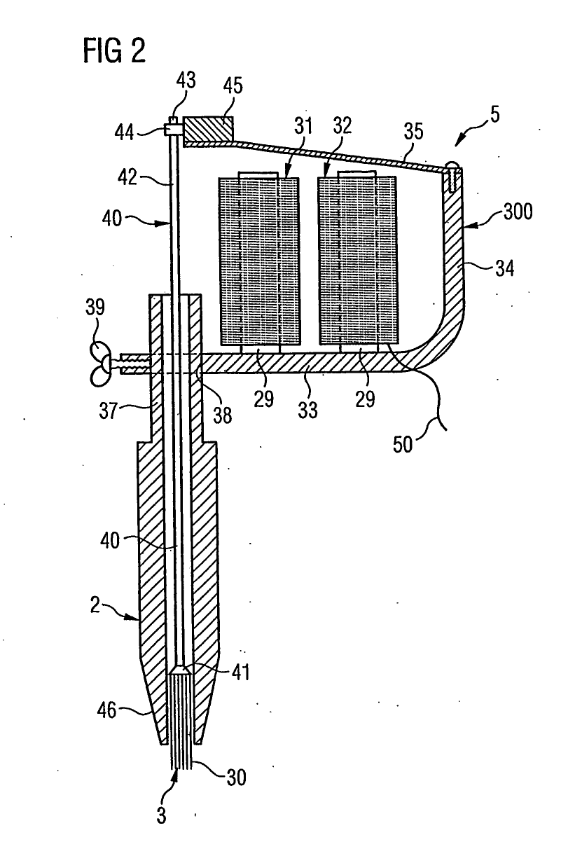

FIG. 2 is a vertical and longitudinal section of a second embodiment mode of one of the means with which to carry out the present method.

A method will be described below wherein the preparation of the invention is mechanically moved to the dye to be removed, which is located under the skin surface. The implement shown in FIG. 1 may be used for that purpose. This implement comprises a housing 1 of optionally round or polygonal cross-section. A holder 2 for the tool 3 of this implement is affixed to the sidewall(s) of the housing 1. In the embodiment mode shown, this holder 2 is round and substantially tubular.

A feedthrough aperture 4 connects the inside of the housing 1 to the inside of the holder 2. The housing 1 receives a drive unit 5 which in this instance includes for instance an electronic motor. The stator of this motor 5 is flanged onto a partition 6 sub-dividing the inside of the housing 1 into two spaces. The first space accommodates the motor 5. Said motor's shaft 7 crosses the partition 6 and bears at its end situated in the second space a flywheel 8. This flywheel 8 is fitted with an excentric pin 9 supporting an elongated transmission element, in this instance a connecting rod 10. An omitted weight is mounted on the flywheel 9 in the vicinity of said pin 9 and, during operation of the means of the invention, will act as an imbalance element.

Advantageously the motor 5 is battery driven and is connected by a cable 50 to an omitted battery. In this manner the present invention need not resort to hardwired power outlets and furthermore the invention eliminates significant heat dissipation, the danger of electric shock injuries, etc.

The connecting rod 10 is fitted at its end facing the drive unit 5 with an eye 11 enclosing the pin 9. The body 12 of the connecting rod 10 crosses the feedthrough aperture 4 and most of the connecting rod 10 is situated in the holder 2. This holder 2 is made in two parts, the first holder part 21 being affixed by means of screws and a flange 13 to the housing 1. The second holder part 22 is joined by a thread 14 to the first holder part 21, and, in the illustrative embodiment mode shown, said second part 22 is screwed on said first part 21. The free end of the second holder part 22 is fitted with a conical transition segment 15, a guide, i.e. a guide bush 16 receiving the actual tool 3 of the present implement merging into the narrowest part of said transition segment 15. The outer edge of the end face 17 of this bush 16 is beveled, whereby this end face 17 is fitted with an external surface in the form of a conical case. This feature allows the implement to operate also when in a slightly slanted position.

When said implement is operating, the flywheel 8 is rotating and the upper end of the connecting rod 10 carries out not only a vertical motion, but also a horizontal one. In order that the connecting rod 10 be able move unhampered in its horizontal displacement, the holder 2 or at least its first part 21 must be of commensurate width. Similar considerations apply to the feedthrough aperture 4. Accordingly the holder 2 also may be designed in a manner to be wider at the top than at the bottom. As regards a vertical longitudinal section situated in a plane perpendicular to the sheet of the drawing, the holder 2 therefore can be substantially V-shaped, its walls running parallel to the above longitudinal section then being able to be configured as shown in FIG. 1.

The lower end, namely the end away from the motor 5, of the connecting rod 10 is fitted with a bush 18 the upper end of which is connected in manner known per se to said rod 10. The lower end of the bush 18 is threaded, preferably on the inside, and this inner, lower end thread of the bush 18 is engaged by the matching thread of the tool 3. This kind of connection between the tool 3 and the drive unit for that tool allows exchanging the tools 3 rapidly and in problem-free manner.

The tool 3 comprises needles 30 and a support for these needles; in the present instance a bolt 20 acts as the needle support. This bolt 20 consists of a threaded part 24 and a seat 25 for the needles 30. The needles 30 are configured in a bundle. The ends of the needles 10 facing the bolt 20, i.e. being situated therein, are firmly joined to each other and accordingly the bundled needles 30 cannot move relative to one another. Adjacent needles 30 are spaced apart and practically run parallel to each other.

The seat 25 is fitted with a cylindrical casing adjoining the threaded part 24. This casing is comparatively long and therefore encloses not only the inside, i.e. upper and mutually connected ends of the needles 30, but also their middle segments. Accordingly only the lower end zones of the needles 30 project from the seat 25. When the connecting rod 10 is extended, the casing of the seat 25 ends in or shortly before the outer mouth of the guide 16. The casing of the seat 25 also acts as a guide for the lower end, namely the end inside the holder 2, of the connecting rod 10. Be it borne in mind that the set of needles 3 per se would be inappropriate for such purposes on account of the elasticity of the individual needles 30 and because of the above mentioned spacing between them.

The skin penetration depth of the needles 30 may be varied when using this implement, for instance by means of several holder parts 22 of the second kind which are of different lengths. The second holder part 22 of the required length is screwed as needed onto the first holder part 21. When the needles 30 all are the same length, their end segments are of different lengths and will project from the guide 16. Another way to change the length of the needle ends projecting from the guide 16 is that tools 3 are available for which the needle ends projecting from the seat 25 are of different lengths. After the second holder part 22 has been screwed in place, the tool 3, which is connected by means of the thread on the bolt 20 to the connecting rod 10, can be screwed off this rod and another tool 3 can be screwed onto it with its needles now of the desired lengths.

FIG. 2 shows a further embodiment mode of the implement of the invention carrying out the present method. In this embodiment mode of said implement, the housing is omitted from the drawing, it being understood that the mechanism shown in FIG. 2 also must be encapsulated. The drive unit 5 is electromagnetic. This drive unit 5 contains two mutually parallel electromagnets 31 and 32 of which the cores 29 are affixed on a common yoke 300. This yoke 300 is substantially L-shaped, the cores 29 being affixed to the horizontal leg 33 of the yoke 300 and near the vertical yoke leg 34. The vertical leg 34 of the yoke 300 is of such length that the armature 35 of the electromagnetic drive 5 can be completely affixed at the end of said yoke. This armature 35 is made of spring steel to allow it to vibrate under the intermittent action of the electromagnets 31 and 32.

The holder 2 for the tool 3 in the present embodiment mode also is elongated and exhibits a merely circular cross-section. The outside diameter of the upper segment of the holder 2 is smaller, as a result of which the holder 2 comprises an offset 37. This holder segment 37 passes through an aperture 38 in the free end zone of the horizontal leg 33. A tightening screw 39 is screwed into the end face of the leg end, the tip of said screw being situated in the aperture 38. The position of the offset holder segment 37 is fixed by means of said screw 39 in the leg aperture 38.

The tool 3 comprises the already above discussed needles 30 which are affixed in a holder 41. The shape of this holder 41 is conical. The needles 30 are affixed for instance by soldering, bonding or the like to the lower, wider end faces of the holder 41. In order that the lower tension rod end be well guided within the holder 2, the borehole diameter in the holder 2 is selected in a way that the wider segment of the conical holder 41 just fits into this borehole. A threaded borehole is present in the end face of the narrow segment of the conical holder 41 and receives the threaded lower end of the tension rod 40. The other upper end of the tension rod 40 is fitted with an eye 43 receiving a pin 44. This pin is affixed by means of the block 45 to the free ends of the armature 35 so that the transmission element 40 and hence also the tool 3 shall be driven by means of said block 45 and the pin 44.

As regards this embodiment mode of the implement of the invention, the tool may be exchanged in two ways. After the holder 2 has been moved into its highest position relative to the horizontal yoke leg 33, the tool 3 is freely accessible in front of the tip 46 of the holder 2 and can be screwed off the tension rod 40 and be replaced by another tool. The other way is to pull the eye 43 at the tension rod 40 together with the tool 3 either upward or downward out of the holder 2.

The length of the needles 30 projecting from the holder 2 may be adjusted in especially simple manner in this latter embodiment mode. It is enough for that purpose merely to loosen the tightening screw 39, to move the holder 2 in either direction in the leg aperture 38 and to again tighten the tightening screw 39.

Tattoo removal is carried out in a manner that a number of skin punctures are carried out at the particular tattooed site. Said punctures are distributed over an area of which the diameter is larger than that of said site. This requirement is met by means of one of the above discussed embodiment modes of the implement of the invention. The said implement is turned ON, whereupon the tool 3 reciprocates in its longitudinal direction. In the process the free end portions of the needles 30 exit from the holder 2. Once the implement has been deposited on the skin surface, the tips of the needles 30 are able to penetrate the skin. As a result the skin punctures so made allow the flushing liquid to reach underneath the skin surface.

The spacings between the individual needles 30 of the tool 3 are selected in a manner that the diameter of the set of needles at the tool 3 is larger than the diameter of the tattoo site. In this way puncture areas with diameters exceeding that of the tattoo site are created in the skin during implement operation. Thereupon the removal liquid is able not only to penetrate directly the particular tattoo site, but also into its vicinity, whereby the tattoo site dye also can be controlled from its surroundings. As already described, the implement is designed to allow puncturing down to controlled depths. Where called for, treatment of the tattooed main sites may be repeated.

The tattoos are removed cosmetically and biologically in the absence of scar formation. The applied liquid being biologically pure, it is highly skin-compatible, entailing no side effect.

The apparatus described above require only brief setup times when changing tools. The tool reciprocating frequency is fairly high while apparatus heat dissipation is minute. The flywheel fitted with an imbalance component assures that the needle punctures are impact-intensive and that the puncture depth shall be uniform. Accordingly calloused skin no longer can adversely affect puncture depth. Thanks to using light-weight metals for the said housing and holder, apparatus weight is comparatively low.

1. A preparation to remove tattoos, comprising:
an aqueous solution of an alkali chloride with a pH value less than 7, and an oil, wherein the concentration of the alkali chloride in the aqueous solution is in the range of 10-20% by weight.

2. The preparation as claimed in claim 1, wherein the alkali chloride is sodium chloride.

3. The preparation as claimed in claim 1, wherein the pH of the solution is less than 4.0, preferably less than 3.4.

4. The preparation as claimed in claim 1, wherein the pH value of the solution is larger than 1.5, preferably larger than 1.8.

5. The preparation as claimed in claim 1 wherein the preparation contains a weak acid with an acid constant 10⁻² mol/ltr>K₁>10⁻⁵ mol/ltr, preferably a fruit acid.

6. The preparation as claimed in claim 1, wherein the preparation contains a citrus fruit juice, preferably pineapple juice.

7. The preparation as claimed in claim 1, wherein the concentration of the alkali chloride in the aqueous solution is within the range of 10-20 and preferably 12-16.5% by weight.

8. The preparation as claimed in claim 1, wherein the preparation additionally contains an oil, preferably coconut oil, or coconut milk.

9. The preparation as claimed in claim 8, wherein the aqueous, acid solution and the oil are mixed in a ratio of 3/1 to 1/1, preferably being two parts water to one part oil.

10. The preparation as claimed in claim 9, wherein the aqueous, acid solution is citrus fruit juice and the oil is coconut milk.

11. The preparation as claimed in claim 1, wherein the preparation additionally contains starch, preferably in the form of potato juice.

12. The preparation as claimed in claim 1, wherein the preparation contains one or several additives, preferably preservatives, oxidizers, whitening agents or buffers.

13. The preparation as claimed in claim 1, wherein the preparation is formulated in a form applicable to the skin, preferably as a cream, an oil, a foam, a gel, an emulsion, a solution or a patch.

14. A method of preparing a tattoo-removing preparation using an aqueous solution of an alkali chloride having a pH value less than 7.0 and an oil ingredient to prepare the tattoo-removing preparation.

15. (canceled)

16. A method of tattoo removal comprising the steps of:
preparing a tatoo-removing preparation wherein the preparation includes an aqueous solution of an alkali chloride with a pH value less than 7 and an oil, and
depositing the preparation on a tattooed skin segment such that skin penetration by the preparation is enhanced.

17. The method as claimed in claim 16, wherein enhancement of preparation penetration is implemented by massaging/kneading the tattooed skin segment and in that the preparation is propagated by diffusion toward a dye-bearing skin layer.

18. The method as claimed in claim 16, wherein the preparation is moved mechanically toward a sub-cutaneous dye to be removed.

19. An implement to carry out a method,
of tattoo removal, wherein a tool (3) is used which introduces a preparation for removing tatoos into a dyed skin layer, in that the tool is fitted with a set of needles (30) and a support (20; 41) for the set of needles, in that the needles (30) are spaced apart from, and end-wise connected to, one another, in that the interconnected needles are affixed to the support (20; 41) and in that such a tool (3) is connected to a drive component in a manner that said tool can be exchanged with another tool (3).

20. The implement as claimed in claim 19, wherein the drive component comprises a drive unit (5) and an elongated transmission element (10, 41), in that said transmission element (10; 41) is connected end-wise to the drive unit and in that the tool ( 3 ) is affixed in an exchangeable manner to the other end of said transmission element.

21. The implement as claimed in claim 20, wherein the transmission element (10; 41) is a connecting rod (10), in that the support for the needles (30) comprises a bolt (20) and in that said bolt is connected for instance by a bush (19) to one end of the connecting rod (10).
